(19) United States
(12) Patent Application Publication
(10) Pub. No.: US 2024/0020652 A1
(43) Pub. Date: Jan. 18, 2024

(54) METHOD FOR ARRANGING APPOINTMENTS IN AN APPOINTMENT SCHEDULE

(71) Applicants: Han-Wen Zheng, Shanghai City (CN); Hon Chen Technology Co., Ltd., Kaohsiung City (TW)

(72) Inventors: Lung-Chi Lee, Kaohsiung City (TW); Lung-I Lee, Kaohsiung City (TW); Han-Wen Zheng, Shanghai City (CN)

(73) Assignees: Han-Wen Zheng, Shanghai City (CN); Hon Chen Technology Co., Ltd., Kaohsiung City (TW)

(21) Appl. No.: 18/348,896

(22) Filed: Jul. 7, 2023

(30) Foreign Application Priority Data
Jul. 13, 2022 (CN) 202210819406.7

(51) Int. Cl. G06Q 10/1093

(52) U.S. Cl. CPC G06Q 10/1095; G16H 40/20

(57) ABSTRACT

A method for arranging appointments in an appointment schedule is to be implemented by a computer storing data that includes a session including a plurality of time slots. The data further includes a plurality of cumulative values corresponding respectively to the time slots and a plurality of maximum values related respectively to the cumulative values. The method includes steps of: in response to receipt of a message for reserving a desired time slot selected from among the time slots, changing the cumulative value and the maximum value that correspond to the desired time slot respectively to a first value and a second value; and performing procedures to update the appointment data related to two groups of the time slots that are in the reservation session and that are respectively after the desired time slot and before the desired time slot.

### CROSS-REFERENCE TO RELATED APPLICATION

This application claims priority to Chinese Invention Patent Application No. 202210819406.7, filed on Jul. 13, 2022.

### FIELD

The disclosure relates to a method for arranging appointments in an appointment schedule.

### BACKGROUND

Conventionally, there are three different methods for making an appointment with a doctor at a clinic or a hospital. The first method is done by a patient reserving a time slot through a reservation machine that is placed in the clinic or the hospital. The second method is done by the patient reserving a time slot on a website or an application program of the clinic or the hospital over the Internet. The third method is done by the patient going directly to a staff member at a counter of the clinic or the hospital, and the staff member at the counter reserving a time slot for the patient manually. The first method and the second method may be too complicated for elderly patients, and thus most elderly patients prefer the third method.

However, when there are too many patients lining up at the counter for making appointments with a doctor, not only would the time taken to make appointments at the counter be very long, but the reserved time slots for these patients may also be packed together and may cause burden on the doctor.

### SUMMARY

Therefore, an object of the disclosure is to provide a method for arranging appointments in an appointment schedule that can alleviate at least one of the drawbacks of the prior art.

According to the disclosure, a method for arranging appointments in an appointment schedule is to be implemented by a computer. The computer stores appointment data that includes a session including a plurality of successive time slots arranged sequentially. The appointment data further includes a plurality of cumulative values corresponding respectively to the successive time slots and a plurality of maximum values related respectively to the cumulative values. The method includes steps of: in response to receipt of a reservation message that is related to reserving a desired time slot selected from among the successive time slots, changing the cumulative value that corresponds to the desired time slot to a first default value, and changing the maximum value that corresponds to the desired time slot to a second default value; and after changing the cumulative value and the maximum value corresponding to the desired time slot, performing a first procedure. The first procedure includes sub-steps of: determining whether there is a posterior time slot that is included in the session including the desired time slot, and that is a first time slot after the desired time slot with the cumulative value equal to the first default value; when determining that the posterior time slot exists, determining whether any of the successive time slots exists between the desired time slot and the posterior time slot; when determining that at least one of the successive time slots exists between the desired time slot and the posterior time slot, reassigning at least one of the cumulative values corresponding respectively to the at least one of the successive time slots that is between the desired time slot and the posterior time slot; and after reassigning the at least one of the cumulative values corresponding respectively to the at least one of the time slots that is between the desired time slot and the posterior time slot, updating, for each of the at least one of the time slots that is between the desired time slot and the posterior time slot, the maximum value corresponding to the time slot based on a greatest one of the at least one of the cumulative values thus reassigned. The method further includes a step of, after changing the cumulative value and the maximum value corresponding to the desired time slot, performing a second procedure. The second procedure includes sub-steps of: determining whether there is a prior time slot that is included in the session including the desired time slot, and that is a last time slot before the desired time slot with the cumulative value equal to the first default value; when determining that the prior time slot exists, determining whether any of the successive time slots exists between the desired time slot and the prior time slot; when determining that at least one of the successive time slots exists between the desired time slot and the prior time slot, reassigning at least one of the cumulative values corresponding respectively to the at least one of the time slots that is between the desired time slot and the prior time slot; and after reassigning the at least one of the cumulative values corresponding respectively to the at least one of the time slots that is between the desired time slot and the prior time slot, updating, for each of the at least one of the time slots that is between the desired time slot and the prior time slot, the maximum value corresponding to the time slot based on a greatest one of the at least one of the cumulative values thus reassigned.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computer for implementing a method for arranging appointments in an appointment schedule according to an embodiment of the disclosure.

FIGS. 2A and 2B cooperatively show a flow chart illustrating a first procedure and a second procedure of the method according to an embodiment of the disclosure.

FIG. 3 is a flow chart illustrating a third procedure of the method according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

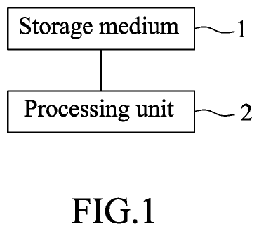

Referring to FIG. 1, a computer is configured to implement a method for arranging appointments in an appointment schedule according to an embodiment of this disclosure. The computer includes a storage medium 1 and a processing unit 2 that is electrically connected to the storage medium 1. The computer may be, but not limited to, a personal computer, a server, a cloud host, a tablet, or a laptop.

The storage medium 1 may be embodied using computer-readable storage medium such as hard disk drive(s), random access memory (RAM), read only memory (ROM), programmable ROM (PROM), firmware, flash memory, etc. The processing unit 2 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), a radio-frequency integrated circuit (RFIC), and/or a system on a chip (SoC), etc.

The storage medium 1 is configured to store appointment data that is related to an appointment schedule. The appointment data includes a plurality of sessions, and each of the sessions includes a plurality of successive time slots that are arranged sequentially. The appointment data further includes, for each of the sessions, a plurality of cumulative values that correspond respectively to the successive time slots included in the session, a plurality of maximum values related respectively to the cumulative values, and a number of available time slots included in the session.

Table 1 below shows an example of the appointment data that is stored in the storage medium 1. It should be noted that the appointment data is not necessarily stored in the storage medium 1 in a format of a table, and Table 1 is provided as an example for illustrative purpose only. The appointment data in Table 1 includes three sessions (i.e., a morning session, an afternoon session, and an evening session). Using the evening session as an example, the evening session includes six time slots between 19:00 to 22:00, and each of the six time slots is 30 minutes in length. All of the six time slots in the evening session are available, so the number of available time slots is assigned as six, the six cumulative values that correspond respectively to the six time slots are assigned in an incremental order from one to six, and the six maximum values that correspond respectively to the six time slots are all equal to six.

To describe in further detail, for any of the six time slots that is itself available, the cumulative value that corresponds thereto indicates a number of consecutively available time slot(s) before said time slot in the evening session, if any, plus one, and for any of the six time slots that is itself unavailable, the cumulative value that corresponds thereto is zero. For example, in Table 1, there are two consecutively available time slots before the time slot of 20:00-20:30, which is itself available, in the evening session, so the cumulative value that corresponds to the time slot of 20:00-20:30 is equal to three. For any of the six time slots that is available, the maximum value that corresponds thereto indicates a number of consecutively available time slot(s) in the evening session, including itself, in a chain of consecutively available time slots in which itself is included, and for any of the six time slots that is unavailable, the maximum value that corresponds thereto is zero. For example, in Table 1, all six time slots in the evening session are available, and thus, for each of the six time slots, there are six consecutive time slots in a chain of consecutively available time slots in which said time slot is included, so the maximum values that correspond respectively to the six time slots are all equal to six.

It should be noted that a number of sessions, a number of time slots, and a time length of each of the time slots should not be limited to the abovementioned example.

TABLE 1

| | | | Number of | |
|---|---|---|---|---|
| | Time | Cumulative | available | Maximum |
| Session | slot | value | time slots | value |
| Morning | 08:00-08:30 | 1 | 8 | 8 |
| | 08:30-09:00 | 2 | | 8 |
| | 09:00-09:30 | 3 | | 8 |
| | 09:30-10:00 | 4 | | 8 |
| | 10:00-10:30 | 5 | | 8 |
| | 10:30-11:00 | 6 | | 8 |
| | 11:00-11:30 | 7 | | 8 |
| | 11:30-12:00 | 8 | | 8 |
| Afternoon | 13:00-13:30 | 1 | 10 | 10 |
| | 13:30-14:00 | 2 | | 10 |
| | 14:00-14:30 | 3 | | 10 |
| | 14:30-15:00 | 4 | | 10 |
| | 15:00-15:30 | 5 | | 10 |
| | 15:30-16:00 | 6 | | 10 |
| | 16:00-16:30 | 7 | | 10 |
| | 16:30-17:00 | 8 | | 10 |
| | 17:00-17:30 | 9 | | 10 |
| | 17:30-18:00 | 10 | | 10 |
| Evening | 19:00-19:30 | 1 | 6 | 6 |
| | 19:30-20:00 | 2 | | 6 |
| | 20:00-20:30 | 3 | | 6 |
| | 20:30-21:00 | 4 | | 6 |
| | 21:00-21:30 | 5 | | 6 |
| | 21:30-22:00 | 6 | | 6 |

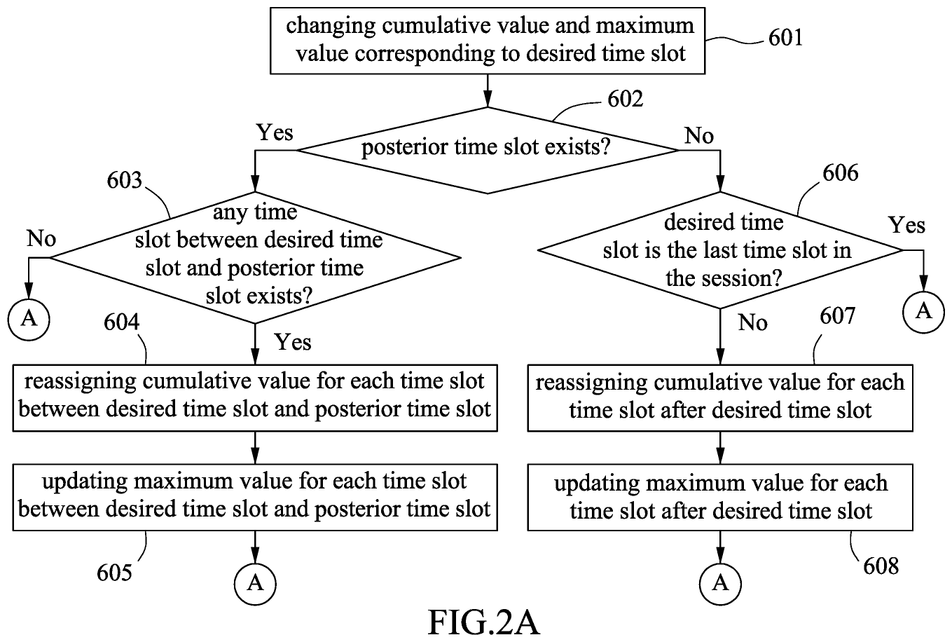

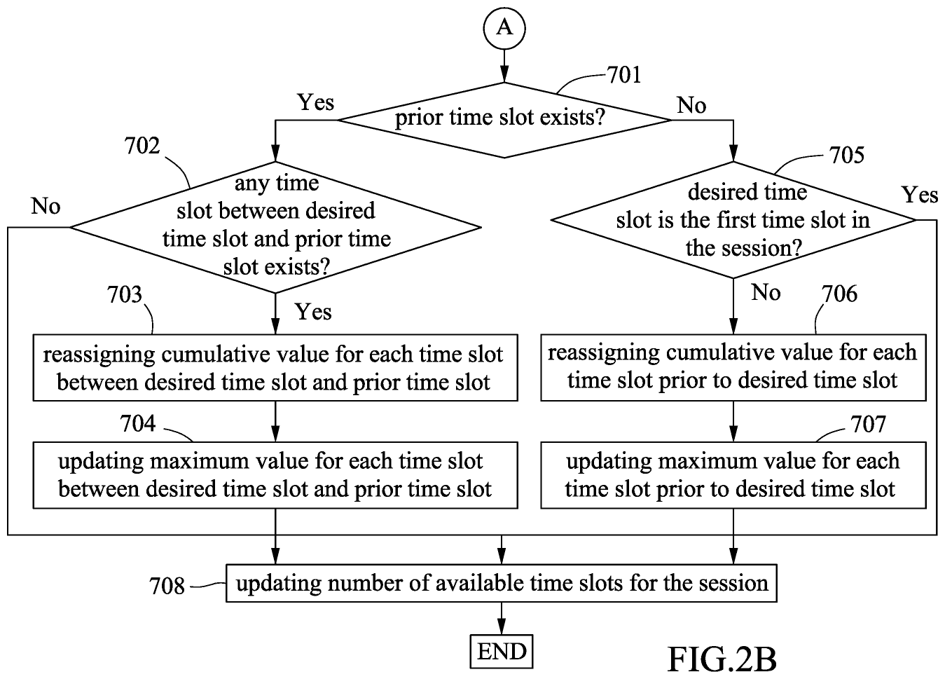

Further referring to FIGS. 2A and 2B, a flow of the method for arranging appointments in the appointment schedule includes steps 601-608 and steps 701-708, where steps 602-608 are part of a first procedure and steps 701-707 are part of a second procedure.

In step 601, in response to receipt of a reservation message that is related to reserving a desired time slot selected from among those of the successive time slots in one of the sessions that are currently available, the processing unit 2 changes the cumulative value that corresponds to the desired time slot to a first default value (e.g., zero), and changes the maximum value that corresponds to the desired time slot to a second default value (e.g., zero).

In the following description, since the method is performed only on the successive time slots that are in the one of the sessions including the desired time slot, only the one of the sessions including the desired time slot (hereinafter referred to as “the reservation session”) is used to describe the method in detail for the sake of simplicity. It should be noted that when the following description refers to one or more of the successive time slots that are after or before the desired time slot, only the one or more of the successive time slots that are in the reservation session are considered.

After step 601, the computer performs the first procedure (i.e., steps 602-608) to update the appointment data related to the successive time slots that are in the reservation session and that are after the desired time slot.

In step 602, the processing unit 2 determines whether there is a posterior time slot that is included in the reservation session and that is a first time slot after the desired time slot with the cumulative value equal to the first default value. If affirmative, the flow proceeds to step 603; otherwise, the flow proceeds to step 606.

In step 603, the processing unit 2 determines whether at least one of the successive time slots is between the desired time slot and the posterior time slot. If the determination made in step 603 is affirmative, the flow proceeds to step 604; otherwise, the first procedure is completed and the flow proceeds to step 701 of the second procedure.

In step 604, the processing unit 2 reassigns at least one of the cumulative values corresponding respectively to the at least one of the successive time slots that is between the desired time slot and the posterior time slot, and then the flow proceeds to step 605. In this embodiment, the at least one of the cumulative values is reassigned in an ascending manner, where the first one of the at least one of the cumulative values is reassigned to one, the second one of the at least one of the cumulative values, if it exists, is reassigned to two, and so on.

In step 605, for each of the at least one of the successive time slots that is between the desired time slot and the posterior time slot, the processing unit 2 updates the maximum value corresponding to the time slot based on a greatest one of the at least one of the cumulative values thus reassigned in step 604. After step 605, the first procedure is completed, and then the flow proceeds to step 701 of the second procedure.

When the determination made in step 602 is negative, the flow proceeds to step 606, where the processing unit 2 determines whether the desired time slot is a last one of the successive time slots in the reservation session. If affirmative, the first procedure is completed and then the flow proceeds to step 701 of the second procedure; otherwise, the flow proceeds to step 607.

In step 607, for each of the successive time slot(s) that is after the desired time slot, the processing unit 2 reassigns the cumulative value corresponding to the time slot, and then the flow proceeds to step 608. Similar to step 604, the cumulative value(s) is(are) reassigned in the ascending manner in step 607, with the first cumulative value being reassigned to one.

In step 608, for any one of the successive time slots that is after the desired time slot, the processing unit 2 updates the maximum value corresponding to the time slot based on a greatest one of the cumulative value(s) thus reassigned in step 607, and the first procedure is completed. Then, the flow proceeds to step 701 of the second procedure.

After the first procedure is completed, the computer performs the second procedure (i.e., steps 701-707) to update the appointment data related to the time slots that are in the reservation session and that are before the desired time slot.

In step 701, the processing unit 2 determines whether there is a prior time slot that is included in the reservation session and that is a last time slot before the desired time slot with the cumulative value equal to the first default value. If affirmative, the flow proceeds to step 702; otherwise, the flow proceeds to step 705.

In step 702, the processing unit 2 determines whether at least one of the successive time slots is between the desired time slot and the prior time slot. If affirmative, the flow proceeds to step 703; otherwise, the second procedure is completed.

In step 703, the processing unit 2 reassigns at least one of the cumulative values corresponding respectively to the at least one of the time slots that is between the desired time slot and the prior time slot, and then the flow proceeds to step 704. Similar to step 604, the at least one of the cumulative values is reassigned in the ascending manner in step 703, with a first one of the at least one of the cumulative values being reassigned to one.

In step 704, for each of the at least one of the time slots that is between the desired time slot and the prior time slot, the processing unit 2 updates the maximum value corresponding to the time slot based on a greatest one of the at least one of the cumulative values thus reassigned in step 703, and the second procedure is completed.

When the determination made in step 701 is negative, the flow proceeds to step 705, where the processing unit 2 determines whether the desired time slot is a first one of the successive time slots in the reservation session. If affirmative, the second procedure is completed; otherwise, the flow proceeds to step 706.

In step 706, for each of the successive time slot(s) that is prior to the desired time slot, the processing unit 2 reassigns the cumulative value corresponding to the time slot, and then the flow proceeds to step 707. Similar to step 607, the at least one of the cumulative values is reassigned in the ascending manner in step 706, with the first cumulative value being reassigned to one.

In step 707, for any one of the successive time slots that is prior to the desired time slot, the processing unit 2 updates the maximum value corresponding to the time slot based on a greatest one of the cumulative value(s) thus reassigned in step 706, and the second procedure is completed.

After both the first procedure and the second procedure are completed, the computer executes step 708. In step 708, the processing unit 2 obtains a count of any of the successive time slots which is included in the reservation session and the cumulative value corresponding to which is not equal to the first default value, and updates the number of available time slots that corresponds to the reservation session based on the count thus obtained, and then the flow of the method ends.

In this embodiment, the second procedure is performed after the first procedure is completed. In some embodiments, the second procedure may be performed before the first procedure is completed, or may be performed simultaneously with the first procedure.

For example, the appointment data is currently as shown in Table 2A below, wherein for the evening session, only the time slot of 19:30-20:00 is unavailable, making the number of available time slots five. In an instance where the reservation message received by the computer is related to reserving the desired time slot of 20:30-21:00, the appointment data after steps 601-608 and steps 701-708 are executed by the computer would become that shown in Table 2B below. In step 601, the cumulative value and the maximum value that correspond to the desired time slot of 20:30-21:00 are changed to the first default value (e.g., zero) and the second default value (e.g., zero), respectively. In the first procedure (i.e., steps 602-608), since there is no posterior time slot after the desired time slot of 20:30-21:00, the cumulative values that correspond respectively to the time slot of 21:00-21:30 and the time slot of 21:30-22:00 are respectively reassigned to one and two (step 607); since a greatest one of the cumulative values thus reassigned in step 607 is equal to two, the maximum values that correspond to the time slot of 21:00-21:30 and the time slot of 21:30-22:00 are both updated to two (step 608). In the second procedure (i.e., steps 701-707), the prior time slot of 19:30-20:00 exists, and the cumulative value that corresponds to the time slot of 20:00-20:30, which is the only time slot between the prior time slot of 19:30-20:00 and the desired time slot of 20:30-21:00, is reassigned to one (step 703); since a greatest one of the cumulative value thus reassigned in step 703 is equal to one, the maximum value that corresponds to the time slot of 20:00-20:30 is updated to one (step 704). Finally, in step 708, the number of available time slots that corresponds to the evening session is updated to four (i.e., the six successive time slots excluding the time slot of 19:30-20:00 and the desired time slot of 20:30-21:00).

TABLE 2A

| | | | Number of | |
|---|---|---|---|---|
| | Time | Cumulative | available | Maximum |
| Session | slot | value | time slots | value |
| Morning | 08:00-08:30 | 1 | 5 | 2 |
| | 08:30-09:00 | 2 | | 2 |
| | 09:00-09:30 | 0 | | 0 |
| | 09:30-10:00 | 0 | | 0 |
| | 10:00-10:30 | 1 | | 2 |
| | 10:30-11:00 | 2 | | 2 |
| | 11:00-11:30 | 0 | | 0 |
| | 11:30-12:00 | 1 | | 1 |
| Afternoon | 13:00-13:30 | 1 | 10 | 10 |
| | 13:30-14:00 | 2 | | 10 |
| | 14:00-14:30 | 3 | | 10 |
| | 14:30-15:00 | 4 | | 10 |
| | 15:00-15:30 | 5 | | 10 |
| | 15:30-16:00 | 6 | | 10 |
| | 16:00-16:30 | 7 | | 10 |
| | 16:30-17:00 | 8 | | 10 |
| | 17:00-17:30 | 9 | | 10 |
| | 17:30-18:00 | 10 | | 10 |
| Evening | 19:00-19:30 | 1 | 5 | 1 |
| | 19:30-20:00 | 0 | | 0 |
| | 20:00-20:30 | 1 | | 4 |
| | 20:30-21:00 | 2 | | 4 |
| | 21:00-21:30 | 3 | | 4 |
| | 21:30-22:00 | 4 | | 4 |

TABLE 2B

| | | | Number of | |
|---|---|---|---|---|
| | Time | Cumulative | available | Maximum |
| Session | slot | value | time slots | value |
| Morning | 08:00-08:30 | 1 | 5 | 2 |
| | 08:30-09:00 | 2 | | 2 |
| | 09:00-09:30 | 0 | | 0 |
| | 09:30-10:00 | 0 | | 0 |
| | 10:00-10:30 | 1 | | 2 |
| | 10:30-11:00 | 2 | | 2 |
| | 11:00-11:30 | 0 | | 0 |
| | 11:30-12:00 | 1 | | 1 |
| Afternoon | 13:00-13:30 | 1 | 10 | 10 |
| | 13:30-14:00 | 2 | | 10 |
| | 14:00-14:30 | 3 | | 10 |
| | 14:30-15:00 | 4 | | 10 |
| | 15:00-15:30 | 5 | | 10 |
| | 15:30-16:00 | 6 | | 10 |
| | 16:00-16:30 | 7 | | 10 |
| | 16:30-17:00 | 8 | | 10 |
| | 17:00-17:30 | 9 | | 10 |
| | 17:30-18:00 | 10 | | 10 |
| Evening | 19:00-19:30 | 1 | 4 | 1 |
| | 19:30-20:00 | 0 | | 0 |
| | 20:00-20:30 | 1 | | 1 |
| | 20:30-21:00 | 0 | | 0 |
| | 21:00-21:30 | 1 | | 2 |
| | 21:30-22:00 | 2 | | 2 |

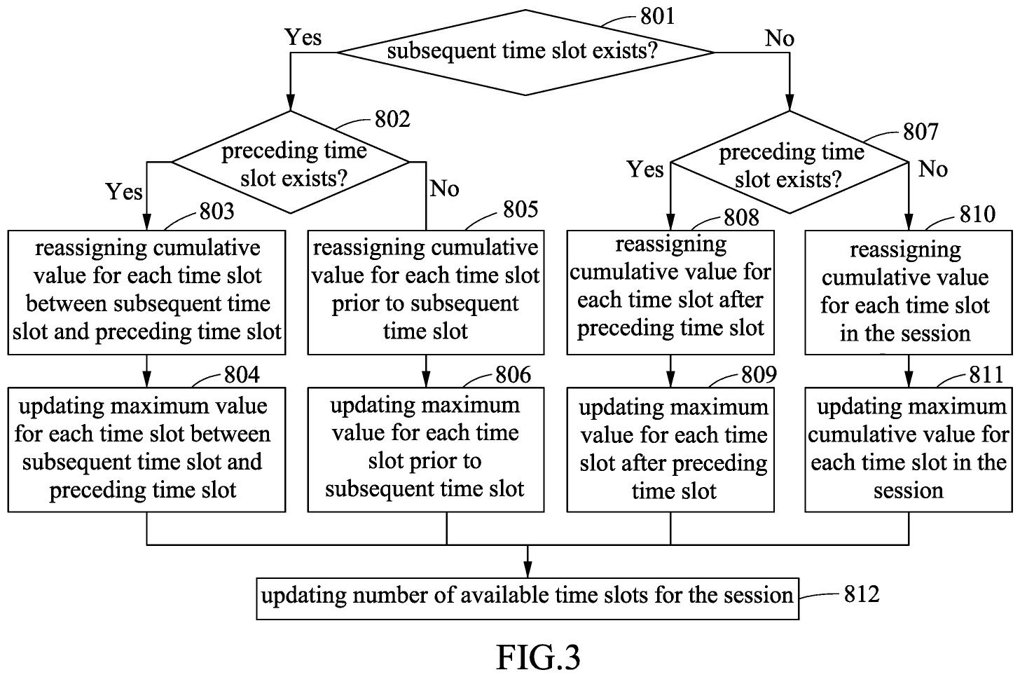

Further referring to FIG. 3, the method for arranging appointments in the appointment schedule further includes a third procedure for cancelling a reservation, where the third procedure includes steps 801 to 812.

When the computer receives a cancellation message that is related to cancelling a reservation of a to-be-cancelled time slot, where the to-be-cancelled time slot is one of the successive time slots that has the cumulative value equal to the first default value, the computer performs the third procedure.

In the following description, since the third procedure is performed only on the successive time slots that are in one of the sessions including the to-be-cancelled time slot, only the one of the sessions including the to-be-cancelled time slot (hereinafter referred to as “the cancellation session”) is used to describe the third procedure in detail for the sake of simplicity. It should be noted that when referring to one or more of the successive time slots that are after or before the to-be-cancelled time slot, only the one or more of the successive time slots that are in the cancellation session are considered.

In step 801, the processing unit 2 determines whether there is a subsequent time slot that is included in the cancellation session and that is a first time slot after the to-be-cancelled time slot with the cumulative value equal to the first default value. If affirmative, the flow of the third procedure proceeds to step 802; otherwise, the flow of the third procedure proceeds to step 807.

In step 802, the processing unit 2 determines whether there is a preceding time slot that is included in the cancellation session and that is a last time slot before the to-be-cancelled time slot with the cumulative value equal to the first default value. If affirmative, the flow of the third procedure proceeds to step 803; otherwise, the flow of the third procedure proceeds to step 805.

In step 803, when determining that both the subsequent time slot and the preceding time slot exist, the processing unit 2 reassigns, for any one of the successive time slots that is between the preceding time slot and the subsequent time slot, the cumulative value corresponding to the time slot, and then the flow of the third procedure proceeds to step 804. More specifically, the to-be-cancelled time slot, which is originally unavailable, will now become available, and the successive time slot(s) between the preceding time slot and the subsequent time slot will be assigned cumulative value(s) in an ascending manner, with the cumulative value of the first time slot among the successive time slot(s) being assigned one.

In step 804, for any one of the successive time slots that is between the preceding time slot and the subsequent time slot, the processing unit 2 updates the maximum value corresponding to the time slot based on a greatest one of the cumulative value(s) thus reassigned in step 803, and then the flow of the third procedure proceeds to step 812.

When determining that the subsequent time slot exists but there is no preceding time slot, in step 805, the processing unit 2 reassigns, for any one of the successive time slots that is prior to the subsequent time slot, the cumulative value corresponding to the time slot, and the flow of the third procedure proceeds to step 806. More specifically, the to-be-cancelled time slot, which is originally unavailable, will now become available, and the successive time slot(s) prior to the subsequent time slot will be assigned cumulative value(s) in an ascending manner, with the cumulative value of the first time slot among the successive time slot(s) being assigned one.

In step 806, for any one of the successive time slots that is prior to the subsequent time slot, the processing unit 2 updates the maximum value corresponding to the time slot based on a greatest one of the cumulative value(s) thus reassigned in step 805, and then the flow of the third procedure proceeds to step 812.

When it is determined in step 801 that the subsequent time slot does not exist, the flow of the third procedure proceeds to step 807, where the processing unit 2 determines whether the preceding time slot exists. If the determination made in step 807 is affirmative, the flow of the third procedure proceeds to step 808; otherwise, the flow of the third procedure proceeds to step 810.

When determining that the subsequent time slot does not exist but the preceding time slot exists, in step 808, the processing unit 2 reassigns, for any one of the successive time slots that is after the preceding time slot, the cumulative value corresponding to the time slot, and then the flow of the third procedure proceeds to step 809. More specifically, the to-be-cancelled time slot, which is originally unavailable, will now become available, and the successive time slot(s) after the preceding time slot will be assigned cumulative value(s) in an ascending manner, with the cumulative value of the first time slot among the successive time slot(s) being assigned one.

In step 809, for any one of the successive time slots that is after the preceding time slot, the processing unit 2 updates the maximum value corresponding to the time slot based on a greatest one of the cumulative value(s) thus reassigned in step 808, and then the flow of the third procedure proceeds to step 812.

When determining that neither the subsequent time slot nor the preceding time slot exists, in step 810, the processing unit 2 reassigns the cumulative values corresponding respectively to all of the successive time slots that are included in the cancellation session in an ascending manner, and then the flow of the third procedure proceeds to step 811.

In step 811, the processing unit 2 updates the maximum values corresponding respectively to the successive time slots that are included in the cancellation session based on a greatest one of the cumulative values thus reassigned in step 810, and then the flow of the third procedure proceeds to step 812.

In step 812, the processing unit 2 obtains another count of any of the successive time slots which is included in the cancellation session and the cumulative value corresponding to which is not equal to the first default value, and updates the number of available time slots that corresponds to the cancellation session based on the another count thus obtained, and the flow of the method ends.

In this embodiment, the subsequent time slot is determined before determining the preceding time slot. In some embodiments, the preceding time slot may be determined before determining the subsequent time slot, or simultaneously with the subsequent time slot.

Taking the appointment data shown in Table 2B as an example, when the cancellation message received by the computer is related to cancelling the reservation of a to-be-cancelled time slot of 19:30-20:00, the appointment data after the third procedure is performed by the computer would become that shown in Table 3 below. The cumulative values that correspond to the time slot of 19:00-19:30, the time slot of 19:30-20:00, and the time slot of 20:00-20:30 (i.e., the successive time slots that are prior to the subsequent time slot of 20:30-21:00) are respectively reassigned as one, two, and three (step 805); since a greatest one of the cumulative values thus reassigned in step 805 is equal to three, the maximum values that correspond to the time slot of 19:00-19:30, the time slot of 19:30-20:00, and the time slot of 20:00-20:30 are all updated to three. Finally, in step 812, the number of available time slots that corresponds to the evening session is updated to five (i.e., the six successive time slots excluding the time slot of 20:30-21:00).

TABLE 3

| | | | Number of | |
|---|---|---|---|---|
| | Time | Cumulative | available | Maximum |
| Session | slot | value | time slots | value |
| Morning | 08:00-08:30 | 1 | 5 | 2 |
| | 08:30-09:00 | 2 | | 2 |
| | 09:00-09:30 | 0 | | 0 |
| | 09:30-10:00 | 0 | | 0 |
| | 10:00-10:30 | 1 | | 2 |
| | 10:30-11:00 | 2 | | 2 |
| | 11:00-11:30 | 0 | | 0 |
| | 11:30-12:00 | 1 | | 1 |
| Afternoon | 13:00-13:30 | 1 | 10 | 10 |
| | 13:30-14:00 | 2 | | 10 |
| | 14:00-14:30 | 3 | | 10 |
| | 14:30-15:00 | 4 | | 10 |
| | 15:00-15:30 | 5 | | 10 |
| | 15:30-16:00 | 6 | | 10 |
| | 16:00-16:30 | 7 | | 10 |
| | 16:30-17:00 | 8 | | 10 |
| | 17:00-17:30 | 9 | | 10 |
| | 17:30-18:00 | 10 | | 10 |
| Evening | 19:00-19:30 | 1 | 5 | 3 |
| | 19:30-20:00 | 2 | | 3 |
| | 20:00-20:30 | 3 | | 3 |
| | 20:30-21:00 | 0 | | 0 |
| | 21:00-21:30 | 1 | | 2 |
| | 21:30-22:00 | 2 | | 2 |

In summary, according to the method provided in this disclosure, in response to receipt of a reservation message, the processing unit 2 determines whether the posterior time slot and the prior time slot exist in the reservation session, and then, according to the determination, reassigns the cumulative value(s), updates the maximum value(s) according to the cumulative value(s) thus reassigned, and updates the number of available time slots that corresponds to the reservation session. Also, in response to receiving a cancellation message, the processing unit 2 determines whether the subsequent time slot and the preceding time slot exist in the cancellation session, and then, according to the determination, reassigns the cumulative value(s), updates the maximum value(s) according to the cumulative value(s) thus reassigned, and updates the number of available time slots that corresponds to the cancellation session. The computer may automatically determine which one of the sessions has more time slots available based on the maximum values corresponding respectively to the successive time slots and/or the numbers of available time slots corresponding respectively to the sessions, thereby effectively reducing time taken to make a reservation for a doctor's appointment and spreading out reserved time slots by choosing to make the reservation in a session with more available time slots.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for arranging appointments in an appointment schedule, the method to be implemented by a computer storing appointment data, the appointment data including a session that includes a plurality of successive time slots arranged sequentially, the appointment data further including a plurality of cumulative values that correspond respectively to the successive time slots and a plurality of maximum values that are related respectively to the cumulative values, the method comprising steps of:
in response to receipt of a reservation message that is related to reserving a desired time slot selected from among the successive time slots, where the desired time slot is available to be reserved, changing the cumulative value that corresponds to the desired time slot to a first default value, and changing the maximum value that corresponds to the desired time slot to a second default value;
after changing the cumulative value and the maximum value that correspond to the desired time slot, performing a first procedure that includes sub-steps of
determining whether there is a posterior time slot that is included in the session including the desired time slot, and that is a first time slot after the desired time slot with the cumulative value equal to the first default value,
when determining that the posterior time slot exists, determining whether any of the successive time slots exists between the desired time slot and the posterior time slot,
when determining that at least one of the successive time slots exists between the desired time slot and the posterior time slot, reassigning at least one of the cumulative values corresponding respectively to the at least one of the successive time slots that is between the desired time slot and the posterior time slot, and
after reassigning the at least one of the cumulative values corresponding respectively to the at least one of the time slots that is between the desired time slot and the posterior time slot, updating, for each of the at least one of the time slots that is between the desired time slot and the posterior time slot, the maximum value corresponding to the time slot based on a greatest one of the at least one of the cumulative values thus reassigned; and
after changing the cumulative value and the maximum value that correspond to the desired time slot, performing a second procedure that includes sub-steps of
determining whether there is a prior time slot that is included in the session including the desired time slot, and that is a last time slot before the desired time slot with the cumulative value equal to the first default value,
when determining that the prior time slot exists, determining whether any of the successive time slots exists between the desired time slot and the prior time slot,
when determining that at least one of the successive time slots exists between the desired time slot and the prior time slot, reassigning at least one of the cumulative values corresponding respectively to the at least one of the time slots that is between the desired time slot and the prior time slot, and
after reassigning the at least one of the cumulative values corresponding respectively to the at least one of the time slots that is between the desired time slot and the prior time slot, updating, for each of the at least one of the time slots that is between the desired time slot and the prior time slot, the maximum value that corresponds to the time slot based on a greatest one of the at least one of the cumulative values thus reassigned.

2. The method as claimed in claim 1, the appointment data further including a number of available time slots included in the session, the method further comprising, after performing the first procedure and the second procedure, a step of:
obtaining a count of any of the successive time slots which is included in the session including the desired time slot and the cumulative value of which is not equal to the first default value, and updating the number of available time slots that corresponds to the session including the desired time slot based on the count thus obtained.

3. The method as claimed in claim 1, wherein the first procedure further includes sub-steps of:
when determining that the posterior time slot does not exist, determining whether the desired time slot is a last one of the successive time slots in the session;
when determining that the desired time slot is not the last one of the successive time slots in the session, for any one of the successive time slots that is after the desired time slot, reassigning the cumulative value corresponding to the time slot; and
after the sub-step of reassigning the cumulative value for any one of the successive time slots that is after the desired time slot, updating, for any one of the successive time slots that is after the desired time slot, the maximum value corresponding to the time slot based on a greatest one of the cumulative value(s) thus reassigned.

4. The method as claimed in claim 1, wherein the second procedure further includes sub-steps of:
when determining that the prior time slot does not exist, determining whether the desired time slot is a first one of the successive time slots in the session;
when determining that the desired time slot is not the first one of the successive time slots in the session, for any one of the successive time slots that is prior to the desired time slot, reassigning the cumulative value corresponding to the time slot; and
after the sub-step of reassigning the cumulative value for any one of the successive time slots that is prior to the desired time slot, updating, for any one of the successive time slots that is prior to the desired time slot, the maximum value corresponding to the time slot based on a greatest one of the cumulative value(s) thus reassigned.

5. The method as claimed in claim 1, further comprising:
when the computer receives a cancellation message that is related to cancelling reservation of a to-be-cancelled time slot, where the to-be-cancelled time slot is one of the successive time slots that has the cumulative value equal to the first default value, performing a third procedure that includes sub-steps of
determining whether there is a subsequent time slot that is included in the session including the to-be-cancelled time slot and that is a first time slot after the to-be-cancelled time slot with the cumulative value equal to the first default value;
determining whether there is a preceding time slot that is included in the session including the to-be-cancelled time slot and that is a last time slot before the to-be-cancelled time slot with the cumulative value equal to the first default value;
when determining that both the subsequent time slot and the preceding time slot exist, for any one of the successive time slots that is between the subsequent time slot and the preceding time slot, reassigning the cumulative value corresponding to the time slot; and
after the sub-step of reassigning the cumulative value for any one of the successive time slots that is between the subsequent time slot and the preceding time slot, updating, for any one of the successive time slots that is between the subsequent time slot and the preceding time slot, the maximum value corresponding to the time slot based on a greatest one of the cumulative value(s) thus reassigned.

6. The method as claimed in claim 5, the appointment data further including a number of available time slots included in the session, the method further comprising, after performing the third procedure, a step of:
obtaining a count of any of the successive time slots which is included in the session including the to-be-cancelled time slot and the cumulative value of which is not equal to the first default value, and updating the number of available time slots that corresponds to the session including the to-be-cancelled time slot based on the count thus obtained.

7. The method as claimed in claim 5, wherein the third procedure further includes sub-steps of:
when determining that the subsequent time slot exists but the preceding time slot does not exist, for any one of the successive time slots that is prior to the subsequent time slot, reassigning the cumulative value corresponding to the time slot; and
after the sub-step of reassigning the cumulative value for any one of the successive time slots that is prior to the subsequent time slot, for any one of the successive time slots that is prior to the subsequent time slot, updating the maximum value corresponding to the time slot based on a greatest one of the cumulative value(s) thus reassigned.

8. The method as claimed in claim 5, wherein the third procedure further includes sub-steps of:
when determining that neither of the subsequent time slot and the preceding time slot exists, reassigning the cumulative values corresponding respectively to the successive time slots that are included in the session including the to-be-cancelled time slot; and
after the sub-step of reassigning the cumulative values corresponding respectively to the successive time slots that are included in the session including the to-be-cancelled time slot, updating the maximum values corresponding respectively to the successive time slots that are included in the session including the to-be-cancelled time slot based on a greatest one of the cumulative values thus reassigned.

9. The method as claimed in claim 5, wherein the third procedure further includes sub-steps of:
when determining that the subsequent time slot does not exist but the preceding time slot exists, for any one of the successive time slots that is after the preceding time slot, reassigning the cumulative value corresponding to the time slot; and
after the sub-step of reassigning the cumulative value for any one of the successive time slots that is after the preceding time slot, for any one of the successive time slots that is after the preceding time slot, updating the maximum value corresponding to the time slot based on a greatest one of the cumulative value(s) thus reassigned.
